SEQUENCE LISTING (8 sequences)

SEQ ID NO: 1 - RNA, 22 nt, Homo sapiens
  hsa-miR-130a
cagugcaauguuaaaagggcau

SEQ ID NO: 2 - RNA, 22 nt, Homo sapiens
  hsa-miR-126
ucguaccgugaguaauaaugcg

SEQ ID NO: 3 - RNA, 22 nt, Homo sapiens
  hsa-miR-21
uagcuuaucagacugauguuga

SEQ ID NO: 4 - RNA, 22 nt, Homo sapiens
  hsa-miR-296-3p
gaggguuggguggaggcucucc

SEQ ID NO: 5 - RNA, 22 nt, Homo sapiens
  hsa-miR-210
cugugcgugugacagcggcuga

SEQ ID NO: 6 - RNA, 21 nt, Homo sapiens
  miR-27a
uucacaguggcuaaguuccgc

SEQ ID NO: 7 - RNA, 22 nt, Homo sapiens
  miR-29a
uagcaccaucugaaaucgguua

SEQ ID NO: 8 - RNA, 23 nt, Homo sapiens
  miR-191
caacggaaucccaaaagcagcug

(19) United States
(12) Patent Application Publication
(10) Pub. No.: US 2022/0151925 A1
(43) Pub. Date: May 19, 2022

(54) METHOD FOR PREDICTING PROANGIOGENIC POTENTIAL OF EXTRACELLULAR VESICLES (EVS)

(71) Applicant: UNICYTE EV AG, Oberdorf (CH)

(72) Inventors: Maria Felice BRIZZI, Torino (IT); Giovanni CAMUSSI, Torino (IT); Claudia CAVALLARI, Torino (IT)

(73) Assignee: UNICYTE EV AG, Oberdorf (CH)

(21) Appl. No.: 17/439,201

(22) PCT Filed: Mar. 13, 2020
(86) PCT No.: PCT/IB2020/052286

§ 371 Date: Sep. 14, 2021

(30) Foreign Application Priority Data
Mar. 15, 2019 (EP) 19163194.4

(51) Int. Cl. A61K 9/127; A61K 38/18; A61K 35/16; A61P 9/10; C12Q 1/6876; C12Q 1/686; G01N 33/68; G01N 33/50; C12Q 1/6804

(52) U.S. Cl. CPC A61K 9/127; A61K 9/1277; A61K 38/1841; A61K 35/16; A61P 9/10; C12Q 1/6876; G01N 2333/495; G01N 33/6863; G01N 33/5032; G01N 33/5085; C12Q 1/6804; C12Q 2600/178; C12Q 2600/158; C12Q 1/686

(57) ABSTRACT

The present invention relates to an in vitro method for predicting the proangiogenic activity of preparations of extracellular vesicles (EVs), preferably blood-derived EVs, wherein the method is based on the combined determination of the content of transforming growth factor beta (TGFβ) and microRNA-130a. Also disclosed is a method of manufacturing a preparation of extracellular vesicles (EVs) predicted to have strong proangiogenic activity and the EVs preparations thereof, which are effective for the therapeutic treatment of ischemic diseases, ischemic injuries and pathological conditions associated with risk of cardiovascular disease, or for use in wound healing.

The present invention relates to a method of predicting proangiogenic activity of extracellular vesicle (EV) preparations, the EV preparations thereof and their therapeutic applications.

Extracellular vesicles (EVs) are small vesicles shed from almost all cell types under both normal and pathological conditions. They mainly include microvesicles, generated by the budding of cell plasma membranes, and exosomes, derived from the endosomal membrane compartment by exocytosis. Recent evidences suggest that EVs could act as mediators of a variety of pathophysiological processes. Increased circulating EVs level has been associated with vascular impairment and hypercoagulability, in particular in patients with diabetes and acute coronary syndrome, suggesting a role in driving cardiovascular diseases. Moreover, an increased level of circulating EVs, mainly originated from platelets and endothelial cells has been proposed as hallmark of cell dysfunction. It has been extensively reported that EVs act as biological active vectors and participate in exchanging information between circulating cells and many cell types including endothelial cells. Indeed, it has been also proposed that EVs derived from platelets play a role in the pathogenesis of atherosclerosis.

EVs act as biological intermediaries mainly by delivering proteins, active lipids and extracellular RNAs, typically referred to as EVs cargo (Pathan M., et al. Vesiclepedia 2019: a compendium of RNA, proteins, lipids and metabolites in extracellular vesicles. (2019) Nucleic Acids Res. 47: D516-D519) However, the most studied EV-mediated biological processes relied on miR transfer. miRs are a class of small noncoding RNAs that post-transcriptionally regulate gene expression. miRs are stably expressed in serum/plasma and their unique expression patterns has been proposed to serve as a disease fingerprint in many clinical settings. Moreover, it has been shown that activated platelets can transfer functional miRs into vascular cells using EVs. This, on turn, regulates ICAM-1 expression and the vascular inflammatory response. Indeed, change in circulating EV cargo has been shown to be associated with endothelial and smooth muscle cell dysfunction in diabetes.

A growing body of evidence suggests that EVs may act as potential therapeutic, diagnostic, and prognostic tools.

The increased risk of cardiovascular events is a common feature of patients suffering of diabetes and obesity. The impaired vessel formation is still considered a relevant mechanism accounting for the abnormal vascular remodeling in these clinical settings. Therefore, to boost neovascularization of damaged tissues remains mandatory to improve patient's outcomes. Different therapeutic approaches have been proposed to improve vascular remodeling in patients with cardiovascular risk factors. However, they failed to provide real benefits, indicating that novel treatment options are still required

WO2018069408 discloses compositions of blood-derived EVs which are characterized by strong proangiogenic activity. Moreover, WO2018069408 teaches a test enabling to measure the proangiogenic potential of EVs, which includes assaying the EVs for their ability to induce cell proliferation and/or a tube-like structure formation in vitro.

The present invention is based on the finding that the composition of the EVs cargo represents a reliable predictive indicator as to whether these vesicles exhibit proangiogenic activity. Surprisingly, the experimental studies carried out by the present inventors, which are illustrated in detail in the experimental section that follows, revealed that EVs possessing proangiogenic activity may be distinguished from inactive vesicles based on the content of transforming growth factor beta (TGFβ) used in combination with the measurement of miR-130a. Indeed, as deduced from ROC analysis, a test based on the combination of the aforementioned measurements exhibits a strong predictive power of discrimination for EVs proangiogenic activity in terms of both sensitivity and specificity, thereby enabling the accurate selection of EVs which are effective in the therapeutic treatment of a disease or injury positively influenced by proangiogenic therapy.

Accordingly, a first aspect of the present invention is a method of predicting whether a composition of extracellular vesicles (EVs) has proangiogenic activity, comprising the steps of:
(a) quantifying the miR-130a microRNA content in the composition of EVs, and
(b) quantifying the transforming growth factor beta (TGFβ) content in the composition of EVs;
(c) determining whether the miR-130a content is above a first predetermined value and the TGFβ content is above a second predetermined value,

wherein:

when the miR-130a content is above said first predetermined value and the TGFβ content is above said second predetermined value, the composition of EVs is predicted to have proangiogenic activity.

As used herein, the term “proangiogenic activity” refers to the stimulation or enhancement of angiogenesis and/or endothelial cell proliferation.

As is further explained in detail below, the inventors have conducted a microarray-based expression profiling of microRNAs (miRNAs) in proangiogenic effective and ineffective EVs isolated from the blood of healthy donors and patients with cardiovascular risk factors, and surprisingly found a significant correlation between the proangiogenic activity of these vesicles and the content of miR-130a. Further studies revealed that EVs possessing proangiogenic activity contain higher amount of the TGFβ protein, as compared to non-active EVs.

Without wishing to be bound by any theory, the inventors believe that the role played by miR-130a in promoting the angiogenic process may be explained by the interaction of this molecule with several genes involved in the angiogenic process, such as KDR, HOXA5, ROCK1, and EPHB6 as revealed by the IPA Ingenuity bioinformatic analysis conducted by the inventors. Moreover, as a result of this analysis, TGFβ and TGFBR1 were also found among the genes under the control of miR-130a, further confirming the cooperation between miR-130a and TGFβ in driving the proangiogenic activity of biologically active EVs.

According to the present invention, the miR-130a nucleotide sequence comprises or consists of nucleotide sequence 5′-CAGUGCAAUGUUAAAAGGGCAU-3′ (SEQ ID NO. 1).

In the method according to the invention, the quantification of miR-130a in the EVs composition is preferably carried out by a nucleic acid-based amplification technique, more preferably by real-time PCR.

In a preferred embodiment, the miR-130 content assessed in the composition of EVs by real-time PCR is measured as a threshold cycle (Ct) value.

Typically, nucleic acid quantification by real-time PCR relies on plotting amplification signal, for example fluorescence, against the number of cycles on a logarithmic scale. As used herein, the term “Ct value” refers to the number of PCR cycles required for the amplification signal to reach an intensity above the background level during the exponential phase of the nucleic acid amplification reaction. Consequently, the Ct value is inversely related to the amount of target nucleic acid initially present in the sample, i.e. the greater the abundance of the target nucleic acid, the smaller the Ct value. Methods for determining the background level in a real-time PCR reaction are well established and known to the person skilled in the art.

More particularly, the present inventors observed that a content of miR130a measured as Ct value of Ct>30 in serum EVs (sEVs) both from healthy subjects and from patients is predictive of proangiogenic ineffective vesicles (<50% as measured in an in vitro potency test).

Accordingly, the EVs composition in the method of the invention is determined to have a miR130a content above a predetermined value. Preferably, the EVs composition in the method of the invention is determined to have a miR130a content as measured as Ct value of Ct less than or equal to 35, more preferably of Ct less than or equal to 33, still more preferably of Ct less than or equal to 30, even more preferably of Ct comprised within the range of from 10 to 29 such as for example 10, 11, 12, 13, 14, 15, 15, 16, 17, 18, 19, 20, 21, 22, 23, 24, 25, 26, 27, 28 or 29.

In a preferred embodiment, the EVs composition in the method of the invention is determined to have a miR130a content as measured as Ct value of Ct<30.

In a preferred embodiment, the amount of miR-130a content in the EVs is determined as a Ct value by applying the 2^{-(ΔCt)} method, which is based on a formula which allows to calculate the relative fold gene expression of samples as described in Livak K J and Schmittgen T D, “Analysis of relative gene expression data using real-time quantitative PCR and the 2(-Delta Delta C(T)) method” (2001) Methods 25: 402-408. As it will be illustrated in detail in the following examples, in order to use the delta-delta Ct method, Ct values measured for all the genes examined and the housekeeping genes have been taken into consideration by the present inventors. Because the whole analysis was based on different housekeeping genes, it was previously made the average of all the housekeeping genes Ct values (Human U& snRNA, RNU43 snoRNA, Hm/Ms/RT U1 snRNA).

In an another preferred embodiment, the quantification of miR-130a content in the EVs is achieved by employing a standard curve of known amounts of miR-130a and interpolating the Ct value determined by real-time PCR in the unknown sample with the standard curve.

While reference is made to real-time PCR, it is understood that other methods of nucleic acid amplification may be used within the scope of this invention, as are known in the art. Such methods include, but are not limited to, nucleic acid sequence-based amplification (NASBA) and digital PCR.

With their studies, the present inventors found that sEVs both from healthy subjects and from patients, which have a content of TGFβ below a predetermined value, preferably below 23 pg/10¹⁰ EVs, are predicted to be proangiogenic inactive.

Therefore, the EVs composition in the method of the invention is determined to have a content of TGFβ above a predetermined value, preferably above a value comprised within the range of from 20 pg/10¹⁰ EVs to 50 pg/10¹⁰ EVs, more preferably above a value within the range of from 23 pg/10¹⁰ EVs to 40 pg/10¹⁰ EVs, even more preferably above a value within the range of from 25 pg/10¹⁰ EVs to 35 pg/10¹⁰ EVs, such as for example 25, 26, 27, 28, 29, 30, 31, 32, 33, 34 or 35 pg/10¹⁰ EVs.

In a preferred embodiment, the EVs composition in the method of the invention is determined to have a content of TGFβ of at least 23 pg/10¹⁰ EVs.

According to the method of the invention, the quantification of TGFβ protein in EVs may be carried out in any suitable manner such as those known in the protein field.

Preferably, the TGFβ content of EVs is measured by an immunoassay. Any suitable immunoassay may be employed, for example, enzyme-linked immunosorbent assay (ELISA), chemiluminescence immunoassay (CLIA), fluorescent immunoassay (FIA), radioimmunoassay (RIAs), precipitation immunoassays, particle immunoassays, competitive binding assays, and the like. More preferably, the immunoassay employed in the method of the invention is an ELISA assay. Obviously, the use of any type of immunoassay format, the selection of which falls within the skills of the ordinary person of skill in the art, is within the scope of the present invention.

In all of the above-described embodiments, it is also preferred that the EVs are derived from human cells.

According to one embodiment of the method of the invention, the proangiogenic activity of the composition of EVs is quantified with an in vitro potency test, which involves testing the EVs by means of a BrdU cell proliferation assay, or a tubulogenesis in vitro assay, or both a BrdU cell proliferation assay and a tubulogenesis in vitro assay.

In a preferred embodiment, the potency test comprises the following steps:

measuring the activity of the EVs composition by a BrdU cell proliferation assay;

measuring the activity of a negative control by a BrdU cell proliferation assay;

measuring the activity of a positive control by a BrdU cell proliferation assay;

calculating the % activity of the composition in the BrdU cell proliferation assay by applying the following formula:

$$\% activity = \frac{composition value - negative control value}{positive crtl value - negative control value} \times 100.$$

According to this embodiment, the method of the invention further comprises step (d) of quantifying the proangiogenic activity of the EVs composition by means of a potency test which comprises the following steps:

testing the composition of EVs by a BrdU cell proliferation assay to obtain a composition value;

testing a negative control by a BrdU cell proliferation assay to obtain a negative control value;

testing a positive control by a BrdU cell proliferation assay to obtain a positive control value;

calculating the % proangiogenic activity of the composition of EVs in the BrdU cell proliferation assay by applying the following formula:

$$\% proangiogenic activity = \frac{composition value - negative control value}{positive crtl value - negative control value} \times 100.$$

The BrdU assay preferably uses HMEC cells seeded in matrigel. In the BrdU cell proliferation assay, serum (preferably 10% serum) is added in the positive control. The negative control is the same medium as the positive control but without the addition of serum. The BrdU assay is preferably based on a concentration of 10000 EVs per target cell.

In another preferred embodiment, the potency test comprises the following steps:

measuring the activity of the EVs composition by a tubulogenesis assay;

measuring the activity of a negative control by a tubulogenesis assay;

measuring the activity of a positive control by a tubulogenesis assay;

calculating the % activity of the composition in the tubulogenesis assay by applying the following formula:

$$\% activity = \frac{composition value - negative control value}{positive crtl value - negative control value} \times 100.$$

According to this embodiment, the method of the invention further comprises step (d) of quantifying the proangiogenic activity of the EVs composition by means of a potency test which comprises the following steps:

testing the composition of EVs by a tubulogenesis assay to obtain a composition value;

testing a negative control by a tubulogenesis assay to obtain a negative control value;

testing a positive control by a tubulogenesis assay to obtain a positive control value;

calculating the % proangiogenic activity of the composition of EVs in the tubulogenesis assay by applying the following formula:

$$\% proangiogenic activity = \frac{composition value - negative control value}{positive crtl value - negative control value} \times 100.$$

The tubulogenesis in vitro assay preferably uses HUVEC cells. In the tubulogenesis in vitro assay, VEGF, preferably 10 ng/ml VEGF, is added to the positive control. The negative control is the same medium as the positive control but without VEGF addition. The tubulogenesis in vitro assay is preferably based on a concentration of 50000 EVs per target cell.

In a more preferred embodiment, both the BrdU cell proliferation assay and the tubulogenesis in vitro assay are used for testing the activity of a given EVs composition against the activity of a positive control, in which case the average % activity values from the BrdU cell proliferation assay and the tubulogenesis assay are compared.

According to this embodiment, the method of the invention further comprises step (d) of quantifying the proangiogenic activity of the composition of EVs by means of a potency test comprising both the BrdU cell proliferation assay and the tubulogenesis in vitro assay as above described.

If the EVs measured activity exceeds a predetermined percentage of the positive control measured activity, for example >50% of the positive control measured activity, the EVs preparation tested is considered as active.

Accordingly, the EVs composition in the method of the invention is preferably determined to have a proangiogenic activity of at least 50%.

Furthermore, the above-described predictive test is suitable to screen for EVs isolated from multiple preparations of body fluid or from the conditioned medium of a cell culture and to identify the pro-angiogenically active preparations for further processing.

Thus, a second aspect of the present invention is a method of manufacturing a preparation of proangiogenic extracellular vesicles (EVs), comprising the steps of:

isolating EVs from multiple preparations of a body fluid or from the conditioned medium of a cell culture;

preparing one or more samples from the isolated EVs at a predetermined concentration of EVs;

predicting the proangiogenic activity of each EVs sample with a method as above described;

selecting the samples wherein miR-130a content is above said first predetermined value, and TGFβ is above said second predetermined value; and optionally

pooling two or more of the active EVs samples, thereby obtaining a preparation of proangiogenic EVs.

EVs derived from serum or other blood components of healthy donors or patients with cardiovascular risk factors are able to induce proangiogenic signals in vitro and in vivo and this effect is not lost even when sEV are pooled.

As mentioned above, with their studies the inventors found that EVs compositions determined to have a content of miR-130a and TGFβ above a first and second predetermined value, respectively, are predicted to possess strong pro-angiogenic properties. These features make them particularly suitable to be employed in the treatment of ischemic diseases, ischemic injuries and pathological conditions associated with risk of cardiovascular disease, or for use in wound healing.

Accordingly, a third aspect of the present invention is a preparation of proangiogenic extracellular vesicles (EVs) wherein the miR-130a content in the preparation measured as Ct value by real-time PCR is Ct less or equal to 35 and the TGFβ content is above a value comprised within the range of from 20 pg/10¹⁰ EVs to 50 pg/10¹⁰ EVs, for use in the therapeutic treatment of a disease or injury positively influenced by proangiogenic therapy or for use in wound healing.

Preferably, the TGFβ content in the preparation of proangiogenic EVs of the invention is above a value comprised within the range of from 23 pg/10¹⁰ EVs to 40 pg/10¹⁰ EVs, more preferably within the range of from 25 pg/10¹⁰ EVs to 35 pg/10¹⁰ EVs, such as for example 25, 26, 27, 28, 29, 30, 31, 32, 33, 34 or 35 pg/10¹⁰ EVs.

In a preferred embodiment, the TGFβ content in the preparation of proangiogenic EVs of the invention is >23 pg/10¹⁰ EVs.

Preferably, the miR-130a content in the preparation of proangiogenic EVs of the invention measured as Ct value by real-time PCR is of Ct less or equal to 33, still more preferably of Ct less or equal to 30, even more preferably of Ct comprised within the range of from 10 to 29 such as for example 10, 11, 12, 13, 14, 15, 15, 16, 17, 18, 19, 20, 21, 22, 23, 24, 25, 26, 27, 28 or 29.

In a preferred embodiment, the miR-130a content in the preparation of proangiogenic EVs of the invention measured as Ct value by real-time PCR is Ct<30.

In a more preferred embodiment, in the preparation of proangiogenic EVs according to the invention the miR-130a content measured as Ct value by real-time PCR is Ct<30 and the TGFβ content is >23 pg/10¹⁰ EVs.

A fourth aspect of the present invention is a preparation of proangiogenic extracellular vesicles (EVs) for use according to the invention as above defined, which is obtainable by the aforementioned method of manufacturing.

The condition associated with risk of cardiovascular disease treated with the EVs preparation according to the present invention is preferably characterized by impaired vascular remodeling, more preferably is obesity, diabetes mellitus, dyslipidemia or hypertension.

In a preferred embodiment, the EVs preparation according to the present invention is suitable for use in the treatment of a disease selected from the group consisting of acute myocardial infarction, acute cerebrovascular disease, acute and chronic peripheral artery disease, acute kidney ischemia, obesity and diabetes mellitus.

Extracellular vesicles are produced by many different cell types—so-called donor cells—and are ubiquitously present in biological fluids, and cellular or tissue cultures. Thus, in accordance with the present invention, the composition of EVs can be obtained from any suitable cell type, preferably from a nucleated mammalian cell, more preferably from a stem cell, even more preferably from an adult stem cell.

Within the context of the present description, the expression “adult stem cell” is intended to mean a stem cell that is isolated from an adult tissue, in contrast with an “embryonic stem cell” which is isolated from the inner cell mass of a blastocyst. Adult stem cells are also known as “somatic stem cells”.

According to an alternative embodiment, the EVs preparation for use according to the invention is isolated from a biological fluid or from a conditioned cell or tissue culture medium.

Preferably, the EVs preparation for use according to the invention is isolated from a blood component, more preferably whole blood, plasma or serum (sEV).

In one embodiment, the proangiogenic EVs are prepared from a blood donation of a healthy donor.

In another embodiment, the proangiogenic EVs are prepared from a blood donation of a patient, more preferably from a patient with cardiovascular risk factors.

In a more preferred embodiment, the proangiogenic EVs prepared from the blood of a patient with cardiovascular risk factors are suitable for use as a medicament for the treatment of the same patient.

Furthermore, in all of the above-described embodiments, the preparation of the invention may be a pharmaceutical preparation, which includes proangiogenic EVs as defined above as well as a pharmaceutically acceptable excipient and/or carrier and/or diluent. The selection of the carrier, vehicle or diluent as well as of any other excipient falls within the skills of the person skilled in the art taking into account, inter alia, the selected pharmaceutical dosage form, administration route and administration regimen, as well as the patient's characteristics and the disease to be treated.

The invention will be better understood from the following examples which are provided by way of illustration only and which make reference to the appended drawings, wherein:

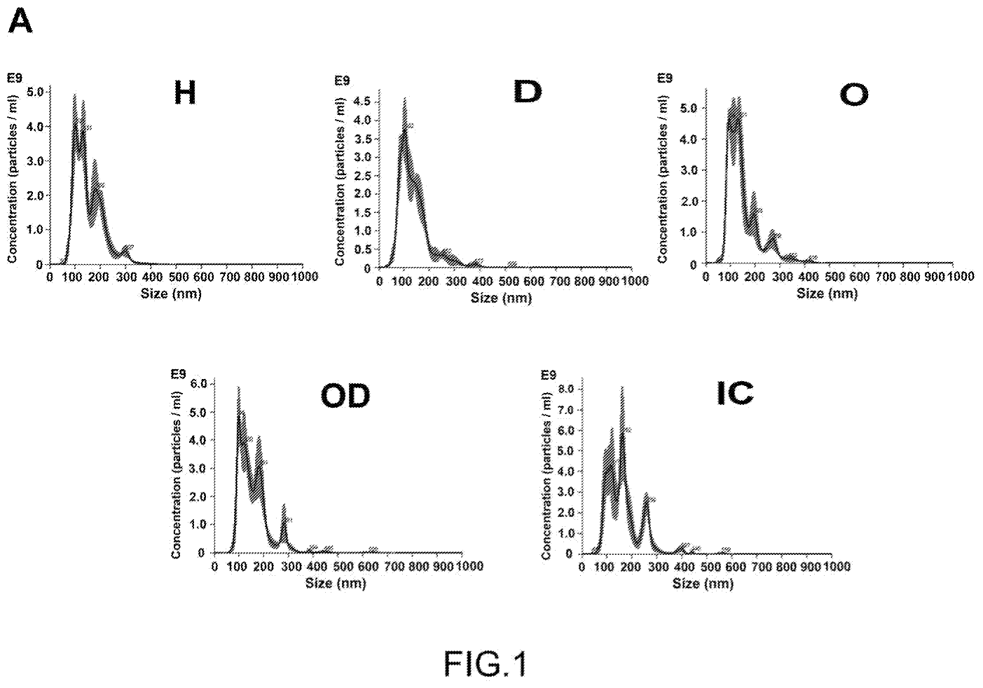

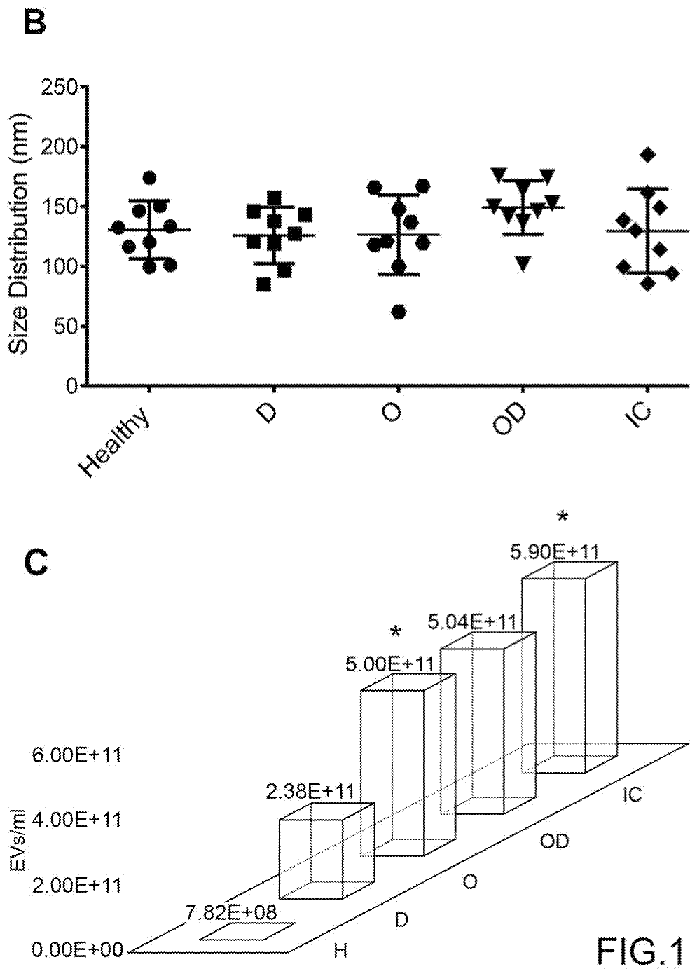

FIG. 1 shows the results of sEV characterization by Nanosight. (A) Representative images of NTA analysis referred to individual groups of patients. (B) Dot plot graph representing NTA size distribution, with mean size value for each individual subject (healthy donor, obese, diabetic, diabetic/obese and ischemic patient). (C) Histogram reporting the number of EVs recovered from serum from individual groups of patients. D=diabetic; O=Obese; OD=obese/diabetic; IC=Ischemic patients. *p<0.05 obese and ischemic patients vs healthy subjects; (One-way ANOVA followed by Multiple Comparison Test) (n=9 patients/group).

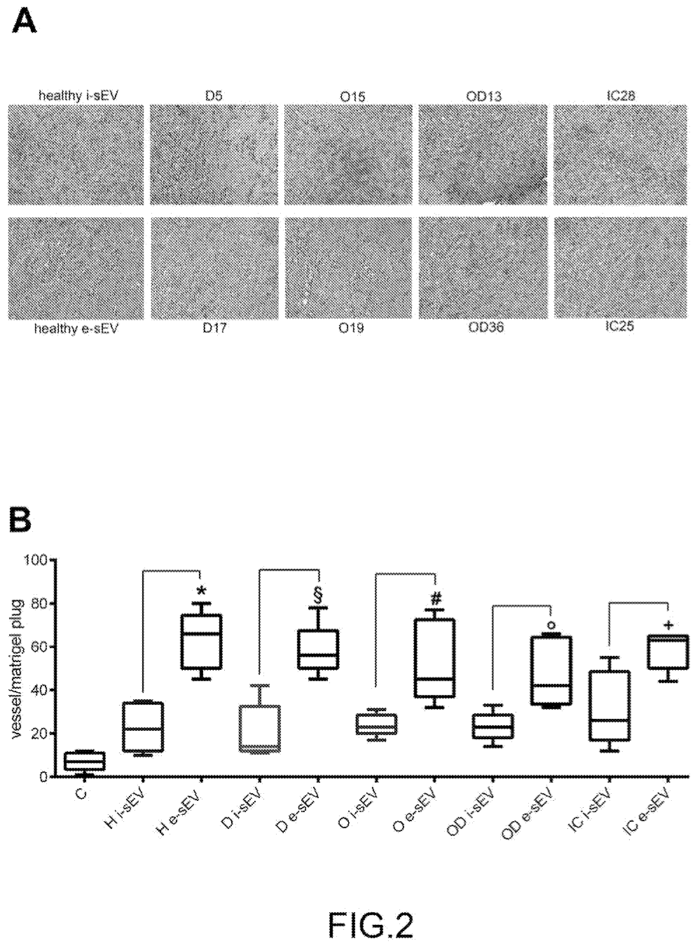

FIG. 2 shows the in vitro and in vivo pro-angiogenic activity of serum EVs from healthy donors and patients (A) Representative micrographs showing vessels formation in response to effective and ineffective sEVs. Each number refers to sEVs prepared from an individual subject (upper panel=ineffective sEVs; lower panel=effective sEVs) (n=3 each group, except for OD the same sample was used in 3 independent experiments). (B) Results of in vivo quantitative analysis of vessel formation. For each experimental condition, vessels were counted in 10 sections of Matrigel. Data show the average number of vessels counted in untreated mice (C) (n=3) or in mice treated with the following preparations of EVs: proangiogenic ineffective sEVs from healthy donors (i-sEVs), proangiogenic effective sEVs from healthy donors (e-sEVs); proangiogenic ineffective sEVs from diabetic patients (D i-sEVs), proangiogenic effective sEVs from diabetic patients (D e-sEVs); proangiogenic ineffective sEVs from obese patients (O i-sEVs), proangiogenic effective sEVs from obese patients (O e-sEVs); proangiogenic ineffective sEVs from diabetic/obese patients (OD i-sEVs), proangiogenic effective sEVs from diabetic/obese patients (OD e-sEVs); proangiogenic ineffective sEVs from ischemic patients (IC i-sEVs), proangiogenic effective sEVs from ischemic patients (IC e-sEVs). *p<0.05 healthy e-sEV vs. healthy i-sEV; § p<0.05 diabetic e-sEV vs. diabetic i-sEV; #p<0.05 obese e-sEV vs. obese i-sEV; °p<0.05 diabetic/obese e-sEV vs. diabetic/obese i-sEV; +p<0.05 ischemic e-sEV vs. ischemic i-sEV ischemic; (One-way ANOVA followed by Multiple Comparison Test). (n=3 each group except for OD the same sample was used in 3 independent experiments). (Original magnification: ×200; scale bar: 12 μm).

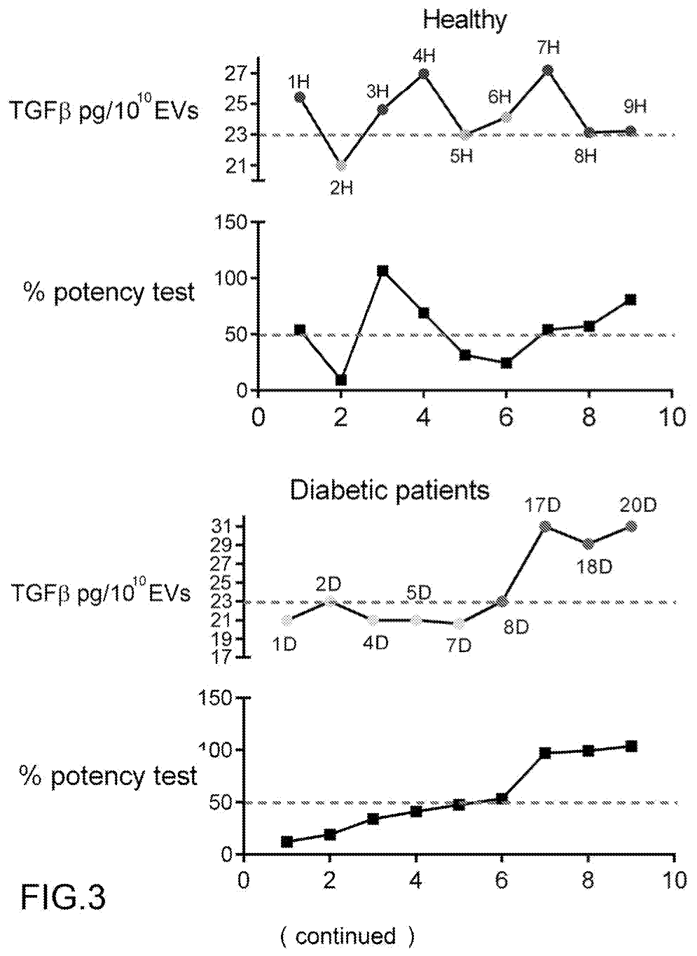

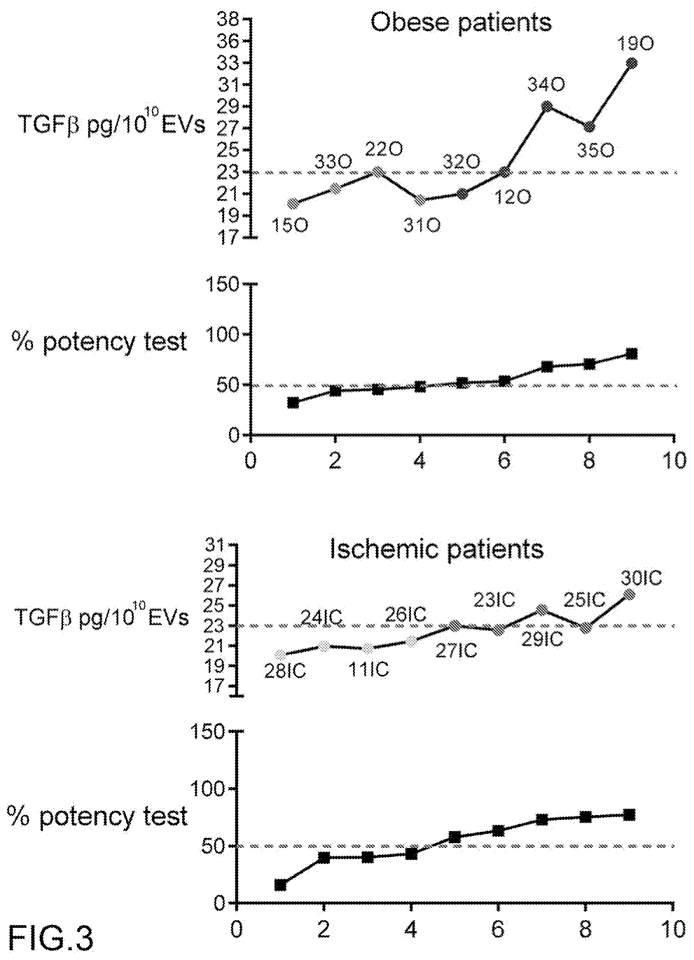

FIG. 3 shows that the proangiogenic activity of sEVs correlates with their TGFβ content. The graphs report the data obtained for samples of serum EVs prepared from individual subjects in each group (healthy donors, diabetic, obese and ischemic patients). For each group of patients, the upper curve is referred to the TGFβ content measured in sEVs as pg/10¹⁰ EVs, while the lower curve is referred to the % of proangiogenic activity as measured in the in vitro potency test. The dotted line indicates the cut-off of TGFβ>23 pg/10¹⁰ EVs for proangiogenic effective and ineffective sEVs. Each number corresponds to an individual patient (n=3 each group).

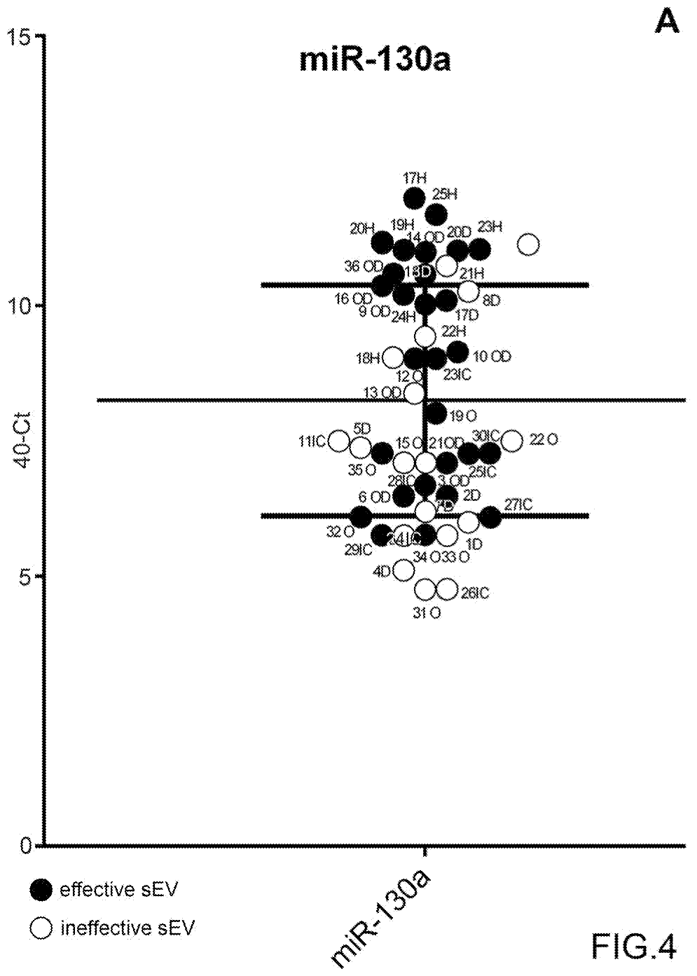

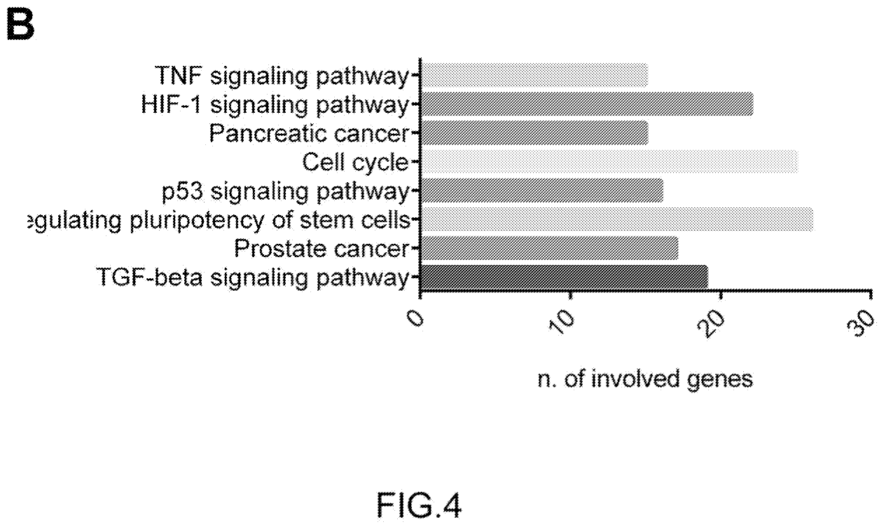

FIG. 4 shows the results of miRNAs expression profiling in sEVs. (A) Distribution of Ct values measured for miR-130a in proangiogenic effective (dark circles) and ineffective (white circles) sEVs from individual patients and healthy subjects. Results are reported as 40-Ct. (B) Network analysis of pathways positively correlated with miR-130a. Data were obtained by DIANA miRpath analysis. Only pathways including at least 15 genes were selected.

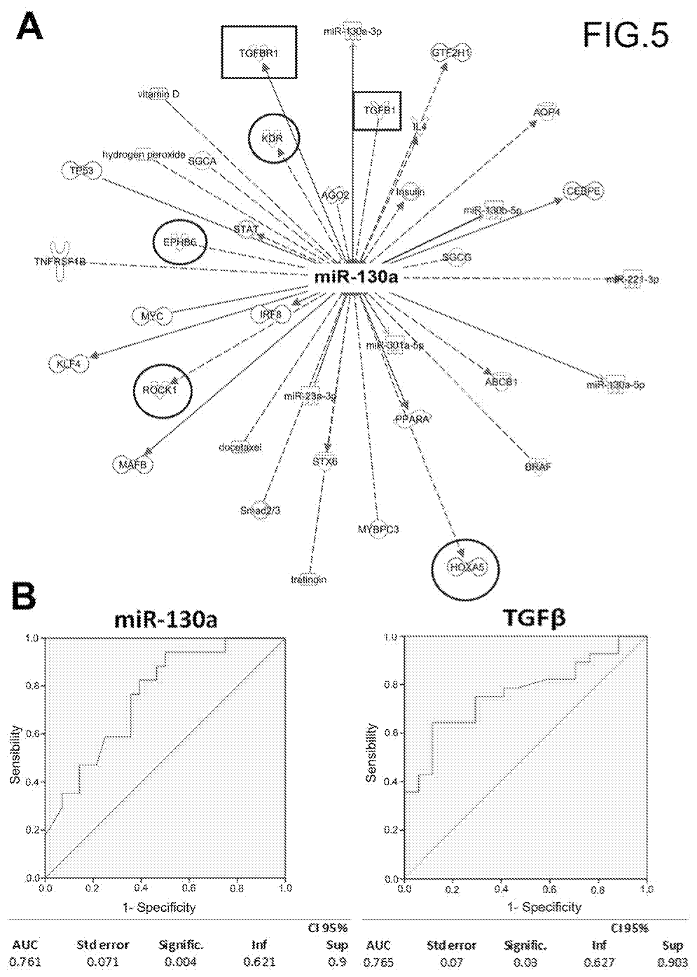

FIG. 5 (A) Network analysis between miR-130a and mRNA targets. Lines represent interactions between genes and miR-130a predicted by the IPA Software: indirect interactions (dotted lines), direct interactions (continuous lines). Squares include TGFβ and TGFBR. Circles include genes involved in angiogenesis (KDR, EPHB6, ROCK1, HOXA5). (B) Receiving Operating Characteristic (ROC) curves and the corresponding area under the curve (AUC) show that miR-130a and TGFβ have predictive ability to discriminate proangiogenic effective sEVs from ineffective vesicles. For ROC analysis, the results obtained for sEVs from all patients and healthy subjects were considered. The AUC values as well as standard errors, p-values, and threshold values are reported in the tables below the ROC curves.

### EXAMPLES

### 1. Method

### 1.1 Patients

In the study carried out by the present inventors, thirty-six patients were included with cardiovascular risk factors and nine sex-matched healthy volunteers. In particular, nine diabetic patients (D: n=9), nine obese patients (O: n=9), nine diabetic and obese patients (OD: n=9), and nine ischemic patients (patients undergoing to surgical treatment for hind limb ischemia) (IC: n=9) were examined. All diabetic patients were not treated with insulin. All human experiments were performed in accordance with European Guidelines and policies and approved by the Ethical Committee of the University of Turin, Italy. Serum from all patients was obtained after admission to the Clinics (D, O, OD) and before surgery for ischemic patients (IC). Informed consent was obtained from all patients. Human serum from healthy donors (n=9) was provided by the Blood Bank of “Città della Salute e della Scienza di Torino”, after informed consent and approval by the internal Review Board of the Blood Bank.

### 1.2. Study Approval

Animal studies were conducted in accordance with the Italian National Institute of Health Guide for the Care and Use of Laboratory Animals (protocol no: 490/2105-PR). Mice were housed according with the Federation of European Laboratory Animal Science Association Guidelines and the Ethical Committee of the University of Turin. All experiments were performed in accordance with relevant guidelines and regulations.

### 1.3. Serum EVs Isolation

Human blood was obtained from healthy and patients donors by venipuncture. A total of 9 ml serum each donor were recovered from each donor and stored at −80° C. After thawing, total EVs were isolated and purified by Ultracentrifugation at 100,000×g for 2 h preceded by a centrifugation at 3000 g to remove debris. Pellets were washed once with PBS and centrifuged at 100.000×g, 4° C. for 1 h. Samples were used fresh or thawed after being stored at −80°.

### 1.4 Nanoparticle Tracking Analysis

sEVs were analyzed by nanoparticle tracking analysis (NTA), using the NanoSight LM10 system (NanoSight Ltd., Amesbury, UK), equipped with a 405 nm laser and with the NTA 2.3 analytic software, to define their dimension and profile. All acquisitions were done with Camera level setting at 14 and for each sample, three videos of 30 s duration were recorded. sEVs were diluted (1:1000) in 1 ml vesicle free physiologic solution (Fresenius Kabi, Runcorn, UK). NTA post-acquisition settings were optimized and maintained constant across samples, and each video was then analyzed to measure EV size, distribution and concentration.

### 1.5 sEVs Angiogenic Assay

Primary macrovascular endothelial cells (ECs) and microvascular endothelial cells (HMEC) were purchased from Lonza (Basel, Switzerland) and cultured as described by the manufacturer's instructions. The in vitro angiogenesis potency test and the in vivo angiogenesis test were performed as previously described (Cavallari C. et al, “Serum-derived extracellular vesicles (EVs) impact on vascular remodeling and prevent muscle damage in acute hind limb ischemia” (2017) Sci Rep. 7(1):8180). Briefly, 5×10⁴ sEVs/target cells were administered throughout the in vitro study. sEVs from single samples were evaluated for their pro-angiogenic activity using BrdU and in vitro tubulogenesis assays. EVs of all the analyzed groups were classified as proangiogenic active or inactive EVs according to a % cut-off value of 50%.

In vivo angiogenesis was assessed by measuring the growth of blood vessels as previously described (Lopatina T. et al, “Platelet-derived growth factor regulates the secretion of extracellular vesicles by adipose mesenchymal stem cells and enhances their angiogenic potential” (2014) Cell Commun Signal. 12:26). Briefly, ECs (1×10⁶ cells/injection) were incubated overnight with sEVs (5×10¹⁰ EVs per 1×10⁶ of ECs). Male severe combined immunodeficiency (SCID) mice (6 weeks old) were then injected subcutaneously. An equal number of non-stimulated ECs was used as a negative control. The Matrigel plugs were recovered on day 7 and fixed and stained using the trichrome stain method. The vessel lumen area was determined as previously described (Lopatina T. et al, “Platelet-derived growth factor regulates the secretion of extracellular vesicles by adipose mesenchymal stem cells and enhances their angiogenic potential” (2014) Cell Commun Signal. 12:26).

### 1.6 TGFβ ELISA Assay

The TGFβ content in the EVs isolated form serum samples of healthy subjects and patients was measured using a solid phase sandwich Enzyme Linked-Immuno-Sorbent Assay (ELISA, Invitrogen Multispecies TGF-β1 kit, Germany) according to the manufacturer's instructions. Experiments were done in triplicate on samples containing 1×10¹¹ EVs. The intensity of the colored product obtained in the assay was determined with an ELISA iMark™ Microplate Absorbance Reader (Bio Rad, Switzerland) with absorbance at 450 nm. The concentration of TGFβ present in the EVs preparations was expressed as pg/10¹⁰ EVs.

### 1.7 miRNA Expression Profiling

The expression profiles of the miRNAs contained in sEVs (so-called miRNome) was assessed by real-time PCR on 1140 microRNAs using miRNome microRNA Profilers QuantiMir (SBI, System Biosciences), according to the protocol recommended by the manufacturer. The kit includes assays in pre-formatted plates for human microRNAs with three endogenous reference RNA as normalization signals (human U6 snRNA, small nucleolar RNA RNU43 and Hm/Ms/Rt U1 snRNA) on each plate.

In brief, 100 ng of RNA has been retrotranscribed using High-Capacity cDNA Reverse Transcription Kit (Applied Biosystems, Foster City, Calif., USA). All qRT-PCR reactions were conducted on the StepOnePlus™ Real-Time PCR System under the following conditions: 15′ at 95° C. (PCR Initial activation step) followed by 3-step cycling (15″ at 94° C., 30″ at 55° C., 30″ at 70° C.) for 40 cycles. In the screening, the miRNome was profiled on sEVs collected from serum of healthy subjects, which had been assessed as proangiogenic active (n=3) and proangiogenic inactive (n=3) with the above described potency test. The Ct values for the miRNAs were extrapolated for each sEVs sample analyzed. A Ct representing the average of Cts from different samples (n=3) of both effective and ineffective sEVs populations was normalized against the endogenous reference RNAs and converted in 2^{-(ΔCt)} values (Livak K J and Schmittgen T D, “Analysis of relative gene expression data using real-time quantitative PCR and the 2(-Delta Delta C(T)) method” (2001) Methods 25: 402-408).

The miRNAs validation was performed on sEVs from healthy donors and patients using the miScript SYBR Green PCR Kit (Qiagen, Valencia, Calif., USA). Briefly, 100 ng of input RNA isolated from sEVs samples were reverse transcribed using the miScript Reverse Transcription Kit and the cDNA thus obtained was used to detect and quantify the miRNAs of interest. Experiments were run in triplicate using 3 ng of cDNA each reaction, as described by the manufacturer's protocol (Qiagen). The following miRNAs were screened in all patient-derived sEVs: miR-126 (SEQ ID NO. 2), miR-21 (SEQ ID NO. 3), miR-296-3p (SEQ ID NO. 4), miR-210 (SEQ ID NO. 5), miR-130a (SEQ ID NO. 1), miR-27a (SEQ ID NO. 6), miR-29a (SEQ ID NO. 7), miR-191 (SEQ ID NO. 8). The amplification data obtained with qRT-PCR were normalized using the RNU6B and the RNU43 reference genes as internal controls. The amplification efficiencies of the target sequence and the endogenous controls were shown to be approximately equal.

### 1.8 Pathway and Target Prediction Analysis of miRNAs EV Content

In order to perform EV miRNAs target prediction and biological pathway enrichment analysis, the web-based program DIANAmirPath was used (Collino F. et al, “Exosome and Microvesicle-Enriched Fractions Isolated from Mesenchymal Stem Cells by Gradient Separation Showed Different Molecular Signatures and Functions on Renal Tubular Epithelial Cells” (2017) Stem Cell Rev. (2):226-43). The algorithm microT-CDS was chosen to predict EV-derived miRNA targets using default threshold (microT=0.8). Only biological pathways showing P value<0.01 to all known Kyoto Encyclopedia of Genes and Genomes (KEGG) pathways were considered as significantly enriched. Ingenuity IPA pathway analysis was used to predict target genes for miR-130a. The inventors set up the miRNA Target Filter tool on IPA (Qiagen: http://www.qiagen-bioinformatics.com/products/ingenuity-pathway-analysis/) to associate miR-130a with predicted mRNA targets.

### 1.9 ROC Analysis

Principal data are presented as means, standard deviations (SD), median and 95% confidence intervals for the two investigated groups “True proangiogenic active sEVs”/“True proangiogenic inactive sEVs”, considered as Reference Standard (RS). In order to evaluate the predictability for miR-130a and TGFβ, the achievement of RS was evaluated using ROC curves (Grund B and Sabin C. “Analysis of biomarker data: logs, odds ratios, and receiver operating characteristic curves” (2010) Curr Opin HIV AIDS 5(6):473-9). The sEVs compositions were classified into the following categories based on the content of miR-130a measured as Ct value and the content of TGFβ, measured as pg/10¹⁰ EVs:

1. sEVs displaying a miR-130a Ct value≥30 were considered proangiogenic ineffective sEVs;
2. sEVs displaying a TGFβ content<23 pg/10¹⁰ EVs were considered proangiogenic ineffective sEVs.

In order to evaluate the ‘goodness’ of the cut-off score based on ROC curve analysis for the above measures to predict “True proangiogenic inactive sEVs” defined in RS, the predictive capacity was evaluated both for each of the two measures separately and by combining the two measures by a ‘Series’ approach (considering as “proangiogenic ineffective sEVs” the sEVs which are proangiogenic ineffective for both measurements and as “NON proangiogenic ineffective sEVs” the vesicles which are “NON proangiogenic ineffective sEVs” for at least one of the two measures).

The analysis was based on Sensitivity (Se), Specificity (Sp), and positive Likelihood ratio (LH+) [Probability of identifying as “proangiogenic ineffective sEVs” a “true proangiogenic ineffective sEVs” compared to a “true proangiogenic effective sEVs”] and relative 95% Confidence Intervals values.

### 1.10 Statistical Analysis

Data were analyzed using the GraphPad Prism 6.0 Demo program. Results are expressed as mean ±SD or ±SEM, unless otherwise reported. Statistical analysis was carried out using 1-way ANOVA, followed by Tukey's post hoc or multiple comparison, Student t tests for 2-group comparison and Newman-Keuls Multiple Comparison Test where appropriate. The cut-off for statistical significance was set at p<0.05 (*p<0.05, **p<0.01, ***p<0.001).

### 2. Results

### 2.1 Characterization of Serum EVs

In the study conducted by the present inventors, nine samples of sEVs derived from healthy individuals and 36 samples of sEVs derived from patients cohorts were examined for their number and size. The distribution of sEVs size did not show any significant difference among healthy individuals and patients (FIGS. 1A and B). The observed average particle size was around 138 nm. The sEVs number in patients was higher than in healthy subjects (FIG. 1C). Significant higher levels of sEVs were detected in obese and ischemic patients (FIG. 1C).

### 2.2 Pro-Angiogenic Activity of Serum EVs Derived from Patients

In order to evaluate in vitro the angiogenic activity of sEVs derived from different patient's groups, a potency test was carried out as described in the Example 1.3 above. The compositions of sEVs showing an average value exceeding 50% were considered as proangiogenic active.

The results of the angiogenic potency test were validated in vivo using proangiogenic effective and ineffective sEVs from different patient's groups (FIG. 2A-B).

### 2.3 TGFβ Content in sEVs and Their Angiogenic Potential

To investigate whether the TGFβ content in sEVs may account for their angiogenic potential, the inventors carried out a ELISA assay on the EVs isolated from serum samples of healthy subjects and patients (diabetic, obese, diabetic/obese and ischemic patients). As shown in FIG. 3, the content of TGFβ measured in the sEVs compositions correlates significantly with the proangiogenic potential of these vesicles in patient cohorts as well as in healthy donors. A cut-off value corresponding to a concentration of TGFβ of 23 pg/10¹⁰ EVs was determined that discriminates proangiogenic effective EVs from ineffective vesicles based on the observation that EVs having a TGFβ content<23 pg/10¹⁰ EVs are more likely to be proangiogenic inactive.

### 2.4 miRNome Profile of sEVs

The miRNome analysis carried out by the present inventors on proangiogenic effective and ineffective sEVs from healthy donors (3 samples/each) led to the identification of eight angio-miRNAs as the most differentially expressed, miR-126, miR-21, miR-296-3p, miR-210, miR-130a, miR-27a, miR-29a, miR-191. In particular, miR-126, miR-130a, miR-27a and 296-3p were up-regulated, while miR-21, miR-29a, miR-191 and miR-210 were down-regulated in sEVs with proangiogenic capability.

To investigate whether the observed difference in miRNAs expression levels in EVs is associated with their functional activity, the inventors carried out a study by comparing the expression of selected miRNAs in sEVs derived from individual healthy donors and patients, with the level of proangiogenic activity of these vesicles as measured with the in vitro potency test. The expression analysis was performed by real time PCR (cut-off Ct value 30). As shown in FIG. 4A, the distribution of the Ct values measured for miR-130a in the EVs from individual subjects (healthy donors and patients) correlates significantly with the results of the angiogenesis potency test performed on these EVs samples. Particularly, it was observed that EVs having a content of miR130a measured as Ct value of Ct>30 have a higher probability of being proangiogenic ineffective.

Interestingly, the present inventors found also an enrichment of miR-210 in sEVs derived from patients. as previous described in Shalaby S M. et al, “Serum miRNA-499 and miRNA-210: A potential role in early diagnosis of acute coronary syndrome” IUBMB Life. 2016; 68(8):673-82. However, no significant correlation was detected between miR-210 content in sEVs and the proangiogenic activity of these vesicles.

DIANA mirpath analysis was interrogated using miR-130a by selecting pathways involving at least 15 genes. Again, among others, a significant enrichment of genes involved in the TGFβ pathway was detected (FIG. 4B).

Network predicted by IPA for miR-130a target genes identified several genes, such as KDR, HOXA5, ROCK1, EPHB6, strongly related to the angiogenic process (FIG. 5A). Moreover, TGFβ and TGFBR1 genes were found among the miR-130a interactors. Overall, the above described results further support the contribution of the TGFβ signaling pathway in sEV-mediated mechanisms of action.

### 2.5 The Content of miR-130a and TGFβ in sEVs Represents a Valuable Predictive Marker to Identify “True Proangiogenic Ineffective” sEVs.

The inventors carried out a Receiver Operator Characteristic (ROC) analysis to assess whether the content of miR-130a and TGFβ in sEVs has the predictive capacity to discriminate between sEVs displaying proangiogenic capability and ineffective vesicles. As deduced from the ROC curves illustrated in FIG. 5B, both miR-130a and TGFβ are good predictive measures of “true proangiogenic ineffective sEVs” identified by RS, showing statistically significant AUC values.

Both measures displayed a good sensitivity to identify as “proangiogenic ineffective” the “true proangiogenic ineffective sEVs” identified by RS. This was particularly evident and further underlined by the LH+=1.88 IC 95% from 1.49 to 2.27, for miR-130a (Se=0.94 IC95% from 0.73 to 0.99) and for TGFβ (Se=0.88 IC95% from 0.66 to 0.97). However, a low specificity value for both measurements was detected (miR-130a: Sp=0.50; TGFβ Sp=0.64).

By combining the two measures ‘in Series’, i.e. considering as “proangiogenic ineffective” those sEVs defined as “proangiogenic ineffective” in both measures, a good level of sensitivity and an increased specificity value were detected (Sp=0.75; Se=0.82). The LH+ value reported in Table 1 below further supports these results.

TABLE 1
Test combining miR-130a and TGFβ1 ‘in series’.
List of values obtained combining the two measures ‘in
Series’ (considering as “proangiogenic ineffective”
the sEVs defined as “proangiogenic ineffective”
in both miR-130a and TGFβ1 measures).

95% Conf. Int.

| Parameters | | Inf | Sup |
|---|---|---|---|
| Se | 0.824 | 0.59 | 0.94 |
| Sp | 0.750 | 0.57 | 0.87 |
| ACC | 0.778 | 0.64 | 0.87 |
| VPP | 0.667 | 0.45 | 0.83 |
| VPN | 0.875 | 0.69 | 0.96 |
| LH+ | 3294118 | 2.62 | 3.97 |
| LH− | 0.235294 | −0.81 | 1.28 |

1. A method for predicting whether a composition of extracellular vesicles (EVs) has proangiogenic activity, the method comprising the steps of:
(a) quantifying the miR-130a microRNA content in the composition of EVs,
(b) quantifying the transforming growth factor beta (TGFβ) content in the composition of EVs; and
(c) determining whether the miR-130a content is above a first predetermined value and the TGFβ content is above a second predetermined value,
wherein:
when the miR-130a content is above said first predetermined value and the TGFβ content is above said second predetermined value, the composition of EVs is predicted to have proangiogenic activity.

2. The method according to claim 1, wherein the miR-130a content is quantified as a Ct value by real-time polymerase chain reaction (real-time PCR) and wherein there is an inverse correlation between the miR-130a content and the Ct value.

3. The method according to claim 2, wherein said first predetermined value is a Ct value<30.

4. The method according to claim 1, wherein the TGFβ content is measured by an immunoassay.

5. The method according to claim 1, wherein said second predetermined value is an amount of TGFβ of 23 pg/10¹⁰ EVs.

6. The method according to claim 1, further comprising the step of
(d) quantifying the proangiogenic activity of the composition of EVs by means a potency test which comprises the following steps:
testing the composition of EVs by a BrdU cell proliferation assay to obtain a composition value;
testing a negative control by the BrdU cell proliferation assay to obtain a negative control value;
testing a positive control by the BrdU cell proliferation assay to obtain a positive control value; and
calculating the % proangiogenic activity of the composition of EVs in the BrdU cell proliferation assay by applying the following formula:
$$\% proangiogenic activity = \frac{composition value - negative control value}{positive crtl value - negative control value} \times 100.$$

7. The method according to claim 1, further comprising the step of
(d) of quantifying the proangiogenic activity of the composition of EVs by a potency test which comprises the following steps:
testing the composition of EVs by a tubulogenesis assay to obtain a composition value;
testing a negative control by the tubulogenesis assay to obtain a negative control value;
testing a positive control by the tubulogenesis assay to obtain a positive control value; and
calculating the % proangiogenic activity of the composition of EVs in the tubulogenesis assay by applying the following formula:
$$\% proangiogenic activity = \frac{composition value - negative control value}{positive crtl value - negative control value} \times 100.$$

8. The method according to claim 1, further comprising the step of
(d) quantifying the proangiogenic activity of the composition of EVs by a potency test comprising
testing the composition of EVs by a BrdU cell proliferation assay to obtain a composition value;
testing a negative control by the BrdU cell proliferation assay to obtain a negative control value;
testing a positive control by the BrdU cell proliferation assay to obtain a positive control value; and
calculating the % proangiogenic activity of the composition of EVs in the BrdU cell proliferation assay by applying the following formula:
$$\% proangiogenic activity = \frac{composition value - negative control value}{positive crtl value - negative control value} \times 100$$
and
testing the composition of EVs by a tubulogenesis assay to obtain a composition value;
testing a negative control by the tubulogenesis assay to obtain a negative control value;
testing a positive control by the tubulogenesis assay to obtain a positive control value; and
calculating the % proangiogenic activity of the composition of EVs in the tubulogenesis assay by applying the following formula:
$$\% proangiogenic activity = \frac{composition value - negative control value}{positive crtl value - negative control value} \times 100.$$

9. The method according to claim 1, wherein the composition of EVs has a proangiogenic activity of at least 50%.

10. The method according to claim 1, wherein the EVs are from human cells.

11. A method for manufacturing a preparation of proangiogenic extracellular vesicles (EVs), the method comprising the steps of:
isolating EVs from multiple preparations of a body fluid or from a conditioned medium of a cell culture;
preparing one or more samples from the isolated EVs at a predetermined concentration of EVs;
predicting the proangiogenic activity of each EVs sample with a method for predicting whether a composition of extracellular vesicles (EVs) has proangiogenic activity, said method comprising
quantifying the miR-130a microRNA content in the composition of EVs,
quantifying the transforming growth factor beta (TGFβ) content in the composition of EVs, and
determining whether the miR-130a content is above a first predetermined value and the TGFβ content is above a second predetermined value.
wherein:
when the miR-130a content is above said first predetermined value and the TGFβ content is above said second predetermined value, the composition of EVs is predicted to have proangiogenic activity;
selecting the samples in which miR-130a content is above said first predetermined value, and TGFβ is above said second predetermined value; and optionally
pooling two or more of active EVs samples,
thereby obtaining a preparation of proangiogenic EVs.

12. The method according to claim 11, wherein the miR-130a content is quantified as a Ct value by real-time PCR and said first predetermined value is a Ct value<30.

13. The method according to claim 11, wherein said second predetermined value is a TGFβ amount of 23 pg/10¹⁰ EVs.

14. The method according to claim 11, wherein the preparation of proangiogenic EVs has a proangiogenic activity of at least 50%.

15. The method according to claim 11, wherein the EVs are from human cells.

16. A method for the therapeutic treatment of a disease or injury positively influenced by proangiogenic therapy or for wound healing in a subject in need thereof, said method comprising administering to said subject a preparation of proangiogenic extracellular vesicles (EVs) obtainable by the method according to claim 11, wherein the miR-130a content in the preparation measured as Ct value by real-time PCR is Ct<30 and the TGFβ content in the preparation is >23 pg/10¹⁰ EVs.

17. A method for the therapeutic treatment of a disease or injury positively influenced by proangiogenic therapy or for wound healing in a subject in need thereof, said method comprising administering to said subject a preparation of proangiogenic extracellular vesicles (EVs) having a miR-130a content measured as Ct value by real-time PCR of Ct<30 and/or a TGFβ content>23 pg/10¹⁰ EVs.

18. The method according to claim 16, wherein the preparation of proangiogenic EVs has a proangiogenic activity of at least 50%.

19. The method according to claim 16, wherein the EVs are derived from a biological fluid or from a conditioned cell or tissue culture medium.

20. The method according to claim 19, wherein the biological fluid is whole blood, plasma, or serum.

21. The method according to claim 20, wherein the EVs are prepared from serum of a healthy donor or from serum of a patient with cardiovascular risk factors.

22. The method according to claim 16, wherein the disease or injury is a vascular disease or injury, or a condition associated with risk of cardiovascular disease.

23. The method according to claim 16, wherein the disease or injury is selected from the group consisting of acute myocardial infarction, acute cerebrovascular disease, acute and chronic peripheral artery disease, acute kidney ischemia, obesity and diabetes mellitus.

24. The method according to claim 4, wherein the TGFβ content is measured by an enzyme-linked immunosorbent assay (ELISA).
